(19) United States
(12) Patent Application Publication
(10) Pub. No.: US 2023/0310751 A1
(43) Pub. Date: Oct. 5, 2023

(54) BLOOD-FILTERING DEVICES FOR USE WITH CLOT TREATMENT SYSTEMS

(71) Applicant: Inari Medical, Inc., Irvine, CA (US)

(72) Inventors: Benjamin Edward Merritt, San Clemente, CA (US); Christopher Andrew Zikry, Northridge, CA (US); John Coleman Thress, Capistrano Beach, CA (US); Parker Ozenne, Aliso Viejo, CA (US); Steven McConnell, Anaheim, CA (US)

(21) Appl. No.: 18/192,855

(22) Filed: Mar. 30, 2023

Related U.S. Application Data
(60) Provisional application No. 63/326,196, filed on Mar. 31, 2022.

(51) Int. Cl. A61M 5/31; A61F 2/01

(52) U.S. Cl. CPC A61M 5/3145; A61F 2/011; A61F 2230/0006

(57) ABSTRACT

Disclosed herein are clot treatment systems with filtering syringes, and associated devices and methods. The filtering syringe can be coupled to a catheter subsystem insertable within a patient. The filtering syringe can aspirate material (e.g., clot material, blood, etc.) from a patient via the catheter subsystem. The filtering syringe can include a plunger assembly, and the plunger assembly can include one or more filters. Each of the filters can be configured to (i) allow at least a first portion of aspirated material (e.g., blood) to pass through at least one of the filters, and (ii) prevent at least a second portion of the aspirated material (e.g., clot material) from passing through at least one of the filters. In some embodiments, the filtering syringe is fluidly coupled to a filtered material subsystem that can fluidly couple the filtering syringe to the patient or another suitable fluid outflow location.

### CROSS-REFERENCE TO RELATED APPLICATIONS

This application claims benefit of U.S. Provisional Application No. 63/326,196, filed Mar. 31, 2022, entitled BLOOD-FILTERING DEVICES FOR USE WITH CLOT TREATMENT SYSTEMS, which is incorporated by reference herein in its entirety.

### TECHNICAL FIELD

The present technology generally relates to blood-filtering devices for use with clot treatment systems, and associated methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

Various devices exist for performing a thrombectomy or removing clot material to reestablish blood flow within a patient. Such devices can remove the target clot material and often additionally remove one or more fluids (e.g., blood) along with the clot material. These devices are often designed to make it difficult to return removed fluids (e.g., blood) to the patient, even when doing so may be advantageous. For example, some existing devices include bypass circuits that can be cumbersome to set up and may require a specially trained user (e.g., a perfusionist) present to operate correctly.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment or clot removal system comprising an aspiration assembly configured in accordance with embodiments of the present technology.

FIG. 2A is a perspective view of a pressure source comprising a vacuum-pressure locking syringe configured in accordance with embodiments of the present technology.

FIG. 2B is a perspective view of a plunger assembly of the vacuum-pressure locking syringe of FIG. 2A in accordance with embodiments of the present technology.

FIG. 2C is an exploded perspective view of the plunger assembly of FIG. 2A in accordance with embodiments of the present technology.

FIGS. 3-7B illustrate various embodiments of filtered material subsystems coupled to the syringe of FIG. 2A and configured in accordance with embodiments of the present technology.

FIG. 8A is a perspective view of another plunger assembly configured in accordance with embodiments of the present technology.

FIG. 8B is a perspective view of a filter assembly configured in accordance with embodiments of the present technology.

FIGS. 9A and 9B are perspective views of another filtering syringe configured in accordance with embodiments of the present technology.

FIG. 10 is a perspective view of another filtering syringe configured in accordance with embodiments of the present technology.

FIG. 11A is a perspective view of another filtering syringe configured in accordance with embodiments of the present technology

FIG. 11B is an enlarged perspective view of a distal portion of the filtering syringe of FIG. 11A, with other portions of the filtering syringe omitted for illustrative clarity.

FIG. 11C is a perspective view of a filter assembly of the filtering syringe of FIG. 11A, configured in accordance with embodiments of the present technology.

FIG. 11D is an enlarged side cross-sectional view of the filtering syringe of FIG. 11A taken along section line 11D-11D in FIG. 11B.

FIG. 12A is a perspective view of a filter assembly of another filtering syringe configured in accordance with embodiments of the present technology.

FIG. 12B is a cross-sectional view of the filter assembly of FIG. 12A taken along section line 12B-12B in FIG. 11B in accordance with embodiments of the present technology.

### DETAILED DESCRIPTION

The present technology is generally directed to clot treatment systems with filters, and associated devices and methods. In some of the embodiments described in detail below, the filters can be part of a filtering syringe. The filtering syringe can be coupled to a catheter subsystem insertable within a patient, such that the filtering syringe can remove (e.g., aspirate) material (e.g., clot material, blood) from a patient via the catheter subsystem. The filtering syringe can include a plunger assembly, and the plunger assembly can include one or more filters. The filters can be configured to (i) allow at least a first portion of material removed from the patient to pass through at least one of the filters, and (ii) prevent at least a second portion of the removed material from passing through at least one of the filters. In some embodiments, for example, the one or more filters are configured to (i) allow blood to pass through at least one of the filters, and (ii) prevent clot material from passing through at least one of the filters, such that the at least one filter can “filter” blood from the material removed from the patient.

In some embodiments, the clot treatment systems further include a filtered material subsystem. The filtered material subsystem can include one or more valves and/or tubing sections that can fluidly couple the filtering syringe and the patient. Additionally, in at least some embodiments, the filtered material subsystem includes a fluid drive element fluidly coupled to the filtering syringe and operable to draw fluid through one or more of the filters, for example, to filter the blood from the clot material within the filtering syringe and/or to otherwise drive flow of the blood through at least part of the filtered material subsystem. In operation, the filtered material subsystem can be configured to (i) capture/receive all or part of the blood within the filtering syringe, and/or (ii) return/reintroduce all or part of the blood within the filtering syringe back to the patient.

In some aspects of the present technology, the filtering syringe can be used to perform multiple material removal (e.g., aspiration) cycles while remaining coupled to the filtered material subsystem and/or the catheter subsystem such that, during one or more of the material removal cycles, at least part of the aspirated material (e.g., blood) removed from the patient can be filtered and/or reintroduced into the patient without disconnecting the filtering syringe from the filtered material subsystem and/or the catheter subsystem, and/or while maintain a generally consistent (e.g., optimal) vacuum power/pressure and/or volumetric flowrate throughout the one or more material removal cycles. In some embodiments, the filtering syringe can be used to perform multiple material removal cycles while remaining fluidly coupled to the catheter system via a single fluid control device. In some aspects, having multiple fluid control devices (e.g., check-valves, Y-connectors, and the like) between a catheter system and a pressure source can reduce the pressure/aspiration power applied by the pressure source to the catheter system. In some embodiments, the filtering syringe can be used to perform one or more material removal cycles while remaining fluidly coupled to catheter and returning at least part of the filtered blood to the patient without using any fluid control devices to control/direct fluid flow between the filtering syringe and the patient. Additionally, or alternatively, at least some of the filtered material subsystems are expected to be safer and/or reduce the likelihood that clot material removed from the patient is accidentally/inadvertently reintroduced into the patient. For example, at least some of the filtered material subsystems can define one or more flow paths configured to direct the flow of the aspirated material to inhibit or prevent the filtered material (e.g., blood) from being reintroduced into the filtering syringe and/or the clot material in the filtering syringe from being driven back into the patient P. In these and other embodiments, at least some of the filtering syringes and/or filtered material subsystems can be configured such that a user can more easily perform one or more tests or other analyses on at least part of the filtered material before or instead of returning the filtered material to the patient.

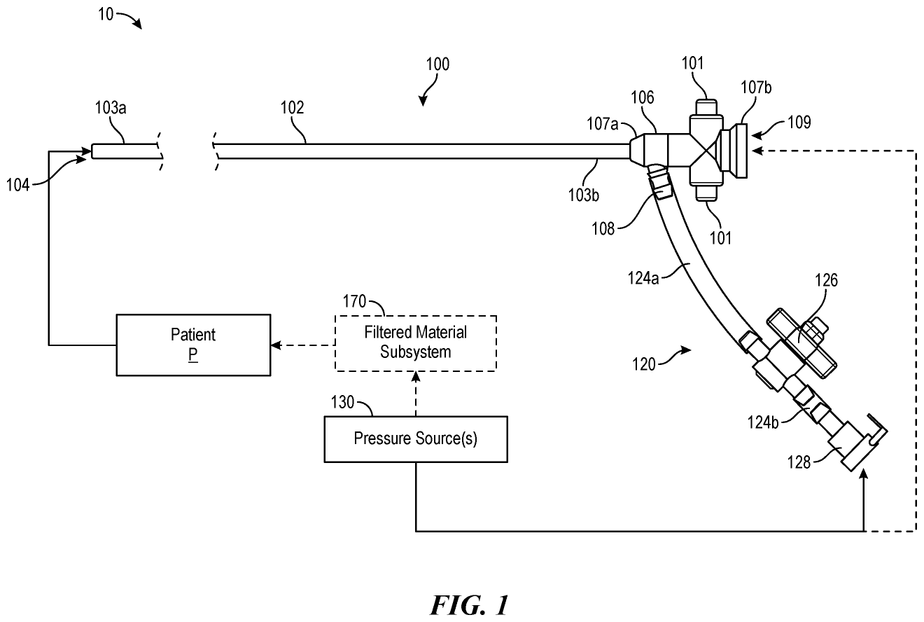

Certain details are set forth in the following description and in FIGS. 1-12B to provide a thorough understanding of various embodiments of the present technology. In other instances, well-known structures, materials, operations, and/or systems often associated with intravascular procedures, blood filtering, clot removal procedures, catheters, and the like are not shown or described in detail in the following disclosure to avoid unnecessarily obscuring the description of the various embodiments of the technology. Those of ordinary skill in the art will recognize, however, that the present technology can be practiced without one or more of the details set forth herein and/or with other structures, methods, components, and so forth. Moreover, although many of the devices and systems are described herein in the context of removing and/or treating aspirated material and/or clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot,” “clot material,” and “aspirated material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as the position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

FIG. 1 is a partially schematic side view of a clot treatment or clot removal system comprising an aspiration assembly 10 (“assembly 10”) configured in accordance with embodiments of the present technology. In the illustrated embodiment, the assembly 10 includes a catheter subsystem 100, a tubing subsystem 120, a pressure source 130, and a filtered material subsystem 170. The catheter subsystem 100 includes a catheter 102 (e.g., an aspiration catheter) comprising an elongated shaft defining a lumen 104 and having a distal portion 103a and a proximal portion 103b. The catheter 102 can be intravascularly advanced to a treatment site within a blood vessel of a patient P such that, for example, the distal portion 103a is positioned in and/or proximate to clot material within the blood vessel. The catheter subsystem 100 further includes a valve 106 that can be integral with or coupled to the proximal portion 103b of the catheter 102.

In general, the clot removal system (i) can include features generally similar or identical to those of the clot removal systems described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein. In some embodiments, the catheter subsystem 100 can include some features that are at least generally similar in structure and function, or identical in structure and function, to those of the catheters disclosed in (i) U.S. Patent Application Publication No. 17/529,018, titled “CATHETERS HAVING SHAPED DISTAL PORTIONS, AND ASSOCIATED SYSTEMS AND METHODS,” and filed Nov. 17, 2021, and/or (ii) U.S. Patent Application Publication No. 17/529,064, titled “CATHETERS HAVING STEERABLE DISTAL PORTIONS, AND ASSOCIATED SYSTEMS AND METHODS,” and filed Nov. 17, 2021, each of which is incorporated herein by reference in its entirety.

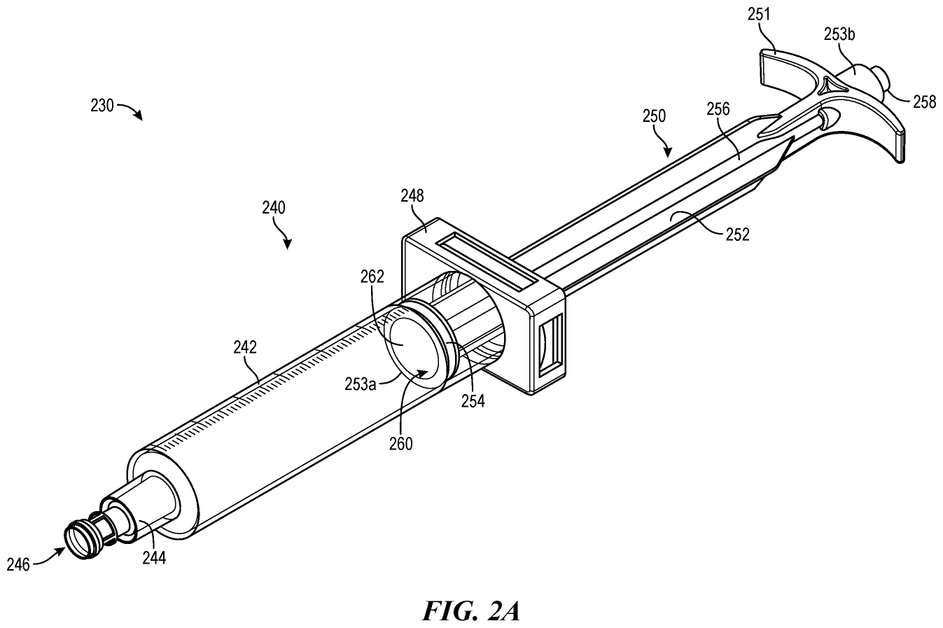

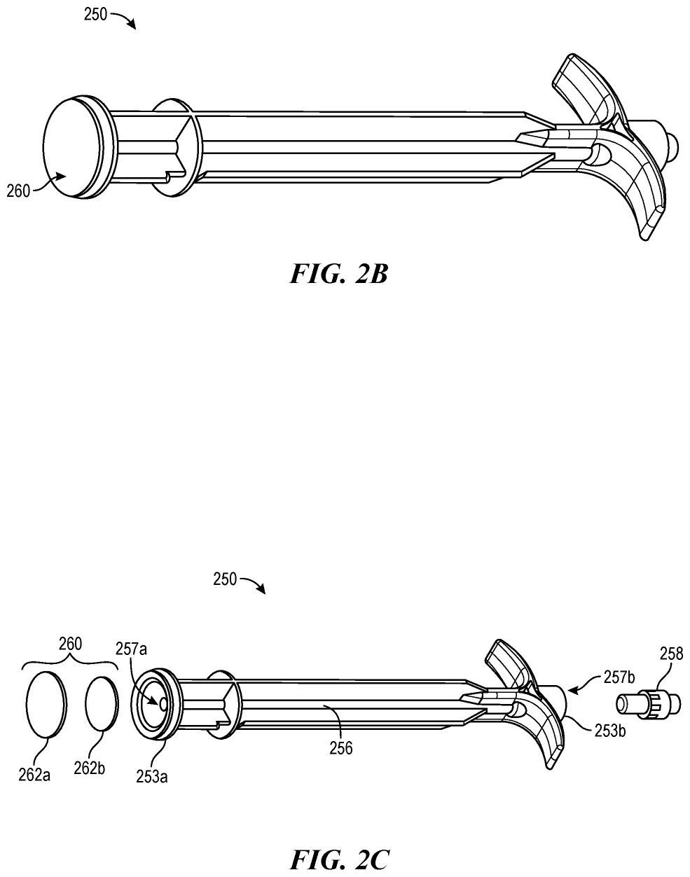

In the illustrated embodiment, the valve 106 includes a distal portion 107a, a proximal portion 107b, and a lumen 109 extending therethrough from the distal portion 107a to the proximal portion 107b. In some embodiments, the valve 106 is a hemostasis valve that is configured to maintain hemostasis during a clot removal procedure by preventing fluid flow in the proximal direction through the valve 106 as various components, such as delivery sheaths, pull members, guidewires, interventional devices, other aspiration catheters, etc., are inserted through the valve 106 to be delivered through the catheter 102 to a treatment site in a blood vessel. The valve 106 further includes one or more branches or side ports 108 configured to fluidly couple the lumen 104 of the catheter 102 to the tubing subsystem 120. In the illustrated embodiment, the valve 106 includes buttons 101 that can be actuated (e.g., depressed) to open a conduit within the lumen 109. In some embodiments, the valve 106 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety. In some embodiments, the proximal portion 107b of the valve 106 is further configured to be detachably coupled (e.g., via a snap-fit arrangement) to a filtering device, such as one or more of the filtering syringes described in greater detail below with reference to FIGS. 2A-2C.

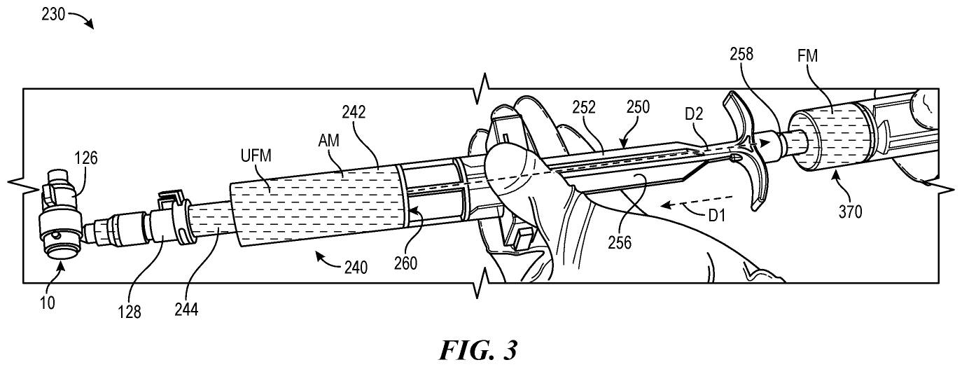

The tubing subsystem 120 fluidly couples the catheter subsystem 100 to the pressure source 130. More specifically, the tubing subsystem 120 can include one or more tubing sections 124 (individually labeled as a first tubing section 124a and a second tubing section 124b), at least one fluid control device 126 (e.g., a valve), and at least one connector 128 for fluidly coupling the tubing subsystem 120 to the pressure source 130 and/or other suitable components. More specifically, in the illustrated embodiment, the fluid control device 126 is a stopcock that is fluidly coupled to (i) the side port 108 of the valve 106 via the first tubing section 124a and (ii) the connector 128 via the second tubing section 124b. In at least some embodiments, the fluid control device 126 is a three-way stopcock, and includes a free port or inlet configured to allow a user to fluidly couple additional devices to the catheter subsystem 100 while the pressure source 130 is also fluidly coupled to the catheter subsystem 100, for example, to reintroduced filtered blood into a patient as described in detail below with reference to FIG. 3. The fluid control device 126 is externally operable by a user to regulate the flow of fluid therethrough and, specifically, from the lumen 104 of the catheter 102 to the pressure source 130. In other embodiments, the fluid control device 126 can be a clamp that can be actuated (e.g., compressed or squeezed by the hand of a user) to partially or fully restrict fluid flow through the first tubing section 124a and/or the second tubing section 124b. In yet other embodiments, the fluid control device 126 can be omitted and its functionality incorporated into the pressure source 130.

The pressure source 130 is configured to generate (e.g., form, create, charge, build up) a vacuum (e.g., negative relative pressure) and store the vacuum for subsequent application to the catheter subsystem 100. Further details of suitable pressure sources are described in detail below with reference to FIGS. 2A-2C and 9A-12B. In some embodiments, the pressure source 130 aspirates fluid (e.g., blood), clot material, and/or other material from the patient P during a clot removal process. In the illustrated embodiment, the pressure source 130 is fluidly coupled to the filtered material subsystem 170. The filtered material subsystem 170 can receive the aspirated fluid, clot material, and/or other material from the pressure source 130 and filter the fluid from the other material. In some embodiments, the filtered material subsystem 170 can be fluidly coupled to the patient P and configured to reintroduce the aspirated fluid (e.g., blood) into the patient. Additional details regarding suitable filtered material subsystems are described in greater detail below with reference to FIGS. 3-7B. Additionally, in at least some embodiments, the filtered material subsystem 170 can be omitted and the pressure source 130 can be used to transfer the aspirated fluid (e.g., blood) to the patient (e.g., intravenously, via the fluid control device 126, via one of the side ports 108, and the like) or any other suitable location.

During operation of the assembly 10, a user can first close the fluid control device 126 before activating the pressure source 130 to build up vacuum pressure within the pressure source 130 (e.g., a vacuum chamber of the pressure source 130). In some embodiments, the user can control or select the volume of the generated vacuum. In this manner, a vacuum is charged within the pressure source 130 before the pressure source 130 is fluidly connected to the catheter subsystem 100. To aspirate the lumen 104 of the catheter 102, the user can open the fluid control device 126 to fluidly connect the pressure source 130 to the catheter subsystem 100 and thereby apply or release the vacuum stored in the pressure source 130 to the lumen 104 of the catheter 102. Opening of the fluid control device 126 instantaneously or nearly instantaneously applies the stored vacuum pressure to the tubing subsystem 120 and the catheter 102, thereby generating suction throughout the catheter 102. In particular, the suction is applied at the distal portion 103a of the catheter 102. In one aspect of the present technology, pre-charging or storing the vacuum before applying the vacuum to the lumen 104 of the catheter 102 is expected to generate greater suction forces (and corresponding fluid flow velocities) at and/or near the distal portion 103a of the catheter 102 compared to simply activating the pressure source 130 while it is fluidly connected to the catheter 102. The suction forces generated by application of the stored vacuum can be used to aspirate or otherwise remove aspirated material from within a blood vessel of a human patient. The aspirated material can be received by the pressure source 130, and at least a portion of the aspirated material can be filtered, transferred to the filtered material subsystem 170, and/or returned to/reintroduced into the patient P or another suitable outflow location.

FIG. 2A is a perspective view of a pressure source 230 comprising a vacuum-pressure locking and/or filtering syringe (“filtering syringe 230”) configured in accordance with embodiments of the present technology. In some embodiments, the filtering syringe 230 can include some features generally similar or identical to those of any one of the syringes described in detail in U.S. patent application Ser. No. 17/396,426, filed Aug. 6, 2021, and titled “AUTOMATICALLY LOCKING VACUUM SYRINGES, AND ASSOCIATED SYSTEMS AND METHODS,” which is incorporated herein by reference in its entirety.

In the illustrated embodiment, the filtering syringe 230 includes a barrel assembly 240 and a filtering plunger assembly 250 (“the plunger assembly 250”). The barrel assembly 240 includes a chamber or barrel 242, a tip 244 extending distally from the barrel 242, and a lumen or bore 246 extending through the tip 244 and the barrel 242. In some embodiments, the barrel assembly 240 includes a locking component 248. The barrel 242 is shown as transparent in FIG. 2A for clarity.

The plunger assembly 250 can include a body or plunger 252 having a first (e.g., distal) end portion 253a defining a distal end terminus of the plunger 252, and a second (e.g., proximal) end portion 253b opposite the first end portion 253a and defining a proximal end terminus of the plunger 252. The plunger assembly 250 can further include a seal 254, a port or valve 258 (such as a Luer-activated valve or any other suitable valve), and a lumen 256 (partially obscured in FIG. 2A) extending at least partially or fully through the plunger 252 between the first end portion 253a and the second end portion 253b. Additionally, the plunger assembly 250 can include a handle 251 to improve user handling of the plunger assembly 250. In the illustrated embodiment, the seal 254 is positioned proximate to the first end portion 253a, and the valve 258 and the handle 251 are positioned proximate to the second end portion 253b. The lumen 256 can be fluidly coupled to the valve 258, and the valve 258 can be fluidly coupled to the filtered material subsystem 170 and/or the patient P (FIG. 1), such that fluid within the lumen 256 can flow to and/or toward the filtered material subsystem 170 and/or the patient P via the valve 258.

The plunger assembly 250 further includes a filter assembly 260. In the illustrated embodiment, the filter assembly 260 is positioned at the first end portion 253a of the plunger 252 and at a distal end portion of the lumen 256. In other embodiments, the filter assembly 260 can be positioned proximate to the second end portion 253b, proximally from the lumen 256, and/or at any other suitable position relative to the lumen 256 and/or within the filtering syringe 230. The filter assembly 260 can include one or more filters 262 (which can also be referred to as “filtering elements,” “filtering stages,” or the like). As described in detail below, fluid (e.g., blood) can be pulled through the filters 262 into the lumen 256 and out of the valve 258.

At least a portion of the plunger assembly 250 is advanceable distally and proximally through the barrel 242. In the illustrated embodiment, the seal 254 sealably engages an interior surface of the barrel 242 and forms a substantially fluid impermeable seal therewith. Referring to both FIGS. 1 and 2A together, the tip 244 can be configured to detachably couple the filtering syringe 230 to the tubing subsystem 120. In the illustrated embodiment, the tip 244 is a Luer connector that can be coupled to the connector 128 and/or the proximal portion 107b of the valve 106 via one or more suitable adaptors. In some embodiments, the barrel 242 can be made of a transparent material that permits a user to visualize material (e.g., aspirated material) within the barrel 242.

With continued reference to FIGS. 1 and 2A together, during operation of the assembly 10, a user can first close the fluid control device 126 and then grip the plunger assembly 250 and/or the barrel assembly 240 to withdraw (e.g., retract) the plunger 252 at least partially out of the barrel 242 to thereby generate a vacuum in the barrel 242. In some embodiments, once the user has withdrawn the plunger 252 to a predetermined volume, the locking component 248 can automatically engage the plunger 252 to lock the plunger 252 relative to the barrel 242. In other embodiments, the plunger 252 can be locked by rotating the plunger 252 relative to the barrel 242 to engage the locking component 248. In some such embodiments, the user can control the volume of the vacuum—by withdrawing the plunger 252 more or less—to provide a desired amount or level of suction/aspiration upon opening of the fluid control device 126. In other embodiments, the filtering syringe 230 may not be a locking syringe, and the user can instead hold the plunger 252 in position relative to the barrel 242. In some embodiments, the syringe has a volume of about 60 cc or less than about 60 cc. With the plunger 252 withdrawn, the user can open the fluid control device 126 to aspirate material (e.g., clot material, bodily fluid(s), and the like) through the lumen 104 and into the barrel 242. The filters 262 of the filter assembly 260 can be selected to allow select material(s) and/or fluid(s) (e.g., one or more vascular fluids, blood, plasma, water, hemoglobin, white blood cells, and the like) aspirated from the patient to pass therethrough. For example, one or more of the filters 262 can (i) allow a first portion of the aspirated material (e.g., blood, blood plasma, and the like) to pass through the filter assembly 260, and (ii) prevent at least a second portion of the aspirated material (e.g., clot material, thrombi, emboli, and the like) from passing through the filter assembly 260. Accordingly, when pressure is applied to the lumen 256, the filter assembly 260 can “filter” or otherwise separate the first portion of the aspirated material (e.g., blood) from the second portion of the aspirated material (e.g., clot material).

FIG. 2B is a perspective view of the plunger assembly 250 of FIG. 2A. FIG. 2C is an exploded perspective view of the plunger assembly 250 of FIG. 2A. Referring to FIGS. 2B and 2C together, in the illustrated embodiment, the filter assembly 260 includes a first filter 262a and a second filter 262b. Each of the filters 262a-b can be formed from a polyester, a polyamide, a hemocompatible material, a combination thereof, and/or any other suitable material. The first and second filters 262a-b can provide multiple stages of filtration to, for example, prolong the life of the filter assembly 260 and/or provide an increased filtering surface area. The first and second filters 262a-b can have the same, generally similar, or different sizes, thicknesses, porosities, and the like. In at least some embodiments, for example, the first filter 262a has a relatively large/coarse porosity of about 200 microns, and the second filter 262b has a relatively small/fine porosity of about 40 microns. In such embodiments, the first filter 262a can be configured to be relatively resilient to negative pressure, large aspirated thrombi, and/or cleaning, and the second filter 262b can be configured to filter blood, microemboli, and the like. In other embodiments, the first filter 262a and/or the second filter 262b can have any other suitable porosity, such as less than 40 microns, more than 200 microns, and/or any porosity therebetween. In these and other embodiments, the first and second filters 262a-b can have any other suitable configuration.

In some embodiments, one or more of the filters 262 can be detachably coupled to the plunger assembly 250 (to, e.g., form the filter assembly 260), such that one or more of the filters 262 can be removed from the plunger assembly 250, such as for cleaning, reuse, and/or disposal. Alternatively or additionally, one or more of the filters 262 can be coupled to, integrally formed with, or otherwise form a single-piece assembly with the plunger 252, such that the plunger 252 can be removed from the barrel 242 to clean, reuse, and/or dispose of the plunger 252 and/or the filter after use. Although the first and second filters 262a-b have a circular shape in the embodiment illustrated in FIG. 2C, in other embodiments one or both of the filters 262a-b can have a disc, pleated, conical, cylindrical, or any other suitable shape. Although in first and second filters 262a-b are positioned adjacent to each other in the embodiment illustrated in FIG. 2C, in other embodiments the first and second filters 262a-b can be spaced apart from each other. In at least some embodiments, for example, the first filter 262a can be positioned proximate the distal end portion 253a of the plunger assembly 250 and the second filter 262b can be positioned proximate the proximal end portion 253b of the plunger assembly 250 opposite the first filter 262a. Although the filter assembly 260 includes two filters in the embodiment illustrated in FIG. 2C, in other embodiments the filter assembly 260 can include more or fewer of the filters 262, such as one, more than two, or any other suitable number of filters 262.

Referring to FIG. 2C, the lumen 256 has a first (e.g., distal) aperture or opening 257a and a second (e.g., proximal) aperture or opening 257b opposite the first opening 257a. In the illustrated embodiment, the first opening 257a is positioned to receive fluid from the barrel 242 (FIG. 2A) through the filter assembly 260, and the second opening 257b is fluidly coupled to the valve 258. In some aspects of the present technology, it is expected that the lumen 256 will be unlikely to substantially clog during use because it receives fluid filtered by the filter assembly 260.

FIGS. 3-7B illustrate embodiments of filtered material subsystems 370-770, respectively, configured in accordance with embodiments of the present technology. Each of the various filtered material subsystems 370-770 can include some features that are generally similar in structure and function, or identical in structure and function, to one another and/or to the corresponding features of the filtered material subsystem 170 described in detail above with reference to FIG. 1 and can operate in a generally similar or identical manner to one another and/or to the filtered material subsystem 170. Moreover, one or more of the different features of the filtered material subsystems 370-770 of the present technology can be combined or omitted.

FIG. 3 is a perspective view of the filtering syringe 230 of FIG. 2A coupled to the assembly 10 of FIG. 1 in accordance with embodiments of the present technology. In the illustrated embodiment, the tip 244 of the barrel assembly 240 is fluidly coupled to the connector 128, and the valve 258 of the plunger assembly 250 is fluidly coupled to a filtered material subsystem 370 generally similar in structure and/or function to the filtered material subsystem 170 of FIG. 1. In the illustrated embodiment, the filtered material subsystem 370 comprises a syringe (“the syringe 370”). In other embodiments, the filtered material subsystem 370 can include a blood bag, a container, and/or any other suitable fluid reservoir.

During a procedure involving the assembly 10, such as a clot treatment and/or a clot removal procedure, a user can open the fluid control device 126 after withdrawing the plunger 252 to charge a vacuum in the barrel 242 to aspirate aspirated material AM into the barrel 242, as described in detail with reference to FIGS. 1 and 2A. The aspirated material AM can include a first (e.g., filterable) portion FM (“the first material FM,” “the filterable material FM,” and the like) and a second (e.g., unfilterable) portion UFM (“the second material UFM,” “the unfilterable material UFM,” and the like). For example, the first material FM can comprise blood and the second material UFM can comprise clot material.

After aspirating the aspirated material AM, the user can close the fluid control device 126 to inhibit the aspirated material AM from flowing back through the fluid control device 126, into the assembly 10, and/or otherwise toward the patient P (FIG. 1). When the aspirated material AM is within the barrel 242, the user can filter the aspirated material AM using the filter assembly 260 to separate all or part of the first material FM from the second material UFM. More specifically, the user can apply a force/pressure to the valve 258 with the syringe 370 to draw the aspirated material AM against the filter assembly 260 and cause at least part of the first material FM to flow through the filter assembly 260 toward the syringe 370. To apply the force/pressure to the valve 258, the user can (i) move the syringe 370 in a first (e.g., distal) direction D1 toward the plunger assembly 250 to depress the plunger 252 through the barrel 242 to drive the aspirated material AM against the filter assembly 260 and the through the lumen 256 to the syringe 370 and/or (ii) actuate the syringe 370 (e.g., by withdrawing a plunger thereof in a second direction D2) to draw the first material FM through the filter assembly 260 without depressing the plunger 252.

Once all or part of the first material FM is within the syringe 370, the user can (i) disconnect the syringe 370 from the valve 258, and/or (ii) return at least a portion of the first material FM (e.g., blood) to the patient. For example, the user can connect the syringe 370 to the fluid control device 126 (e.g., the free port or inlet, described above with reference to FIG. 1) and actuate the syringe 370 to inject all or part of the first material FM within the syringe 370 into the catheter 102 (FIG. 1) and the patient. As another example, the user can use the syringe 370 to inject all or part of the first filtered material FM into the patient intravenously, via one or more of the side ports 108 (FIG. 1), and/or using any other suitable method or technique. Additionally, or alternatively, the user can (i) disconnect the filtering syringe 230 from the assembly 10, (ii) remove all or part of the remaining aspirated material AM and/or the second material UFM from the barrel 242, and/or (iii) use the filtering syringe 230 to perform one or more subsequent aspirations to remove additional material from the patient. In some embodiments, for example, the user can flush all or part of the aspirated material AM from the filtering syringe 230 via the valve 258 by, for example, fluidly coupling the valve 258 to a fluid source and flowing fluid at least partially through the filtering syringe 230 and toward and/or out of the tip 244. In other embodiments, the user can place the tip 244 of the filtering syringe 230 into a bowl containing a saline solution, or any other suitable washing/flushing solution, withdraw the plunger 252 to pull at least a portion of the saline solution into the barrel 242 (e.g., to mix with any aspirated material AM contained therein), and then depress the plunger 252 to expel all or part of the aspirated material AM and/or the saline solution from the barrel 242. In these and other embodiments, the user can de-air the filtering syringe 230 before aspirating material from the patient. For example, the user can use the syringe 370 to (i) inject liquid (e.g., saline) into the filtering syringe 230 and/or (ii) draw fluid (e.g., air, liquid, etc.) from within the filtering syringe via, for example, the valve 258 on the filtering syringe 230. Additionally, or alternatively, the user can de-air the syringe 370 before reintroducing the first filtered material FM into the patient, for example, by actuating the plunger of the syringe 370 to remove air bubbles from the syringe's barrel.

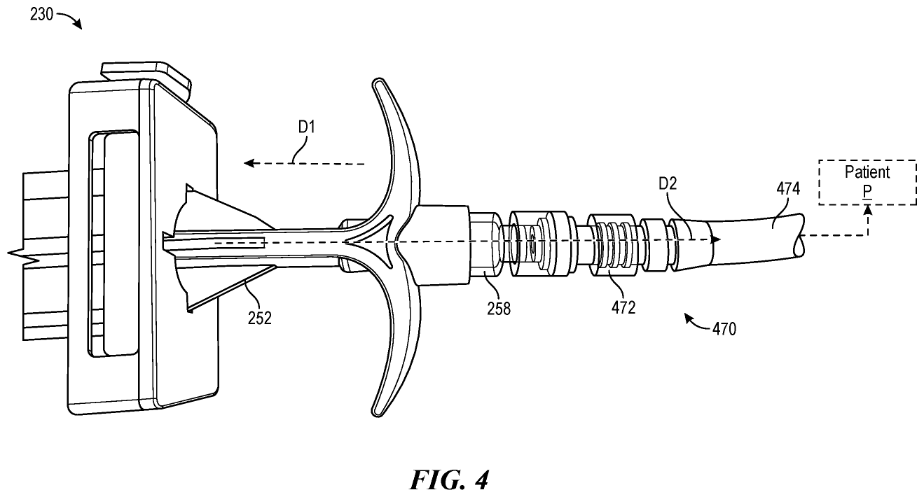

FIG. 4 is an enlarged side view of the filtering syringe 230 of FIG. 2A coupled to a filtered material subsystem 470 in accordance with embodiments of the present technology. In the illustrated embodiment, the filtered material subsystem 470 includes a fluid control device 472 and a tubing section 474. The tubing section 474 can fluidly couple the filtering syringe 230, via the fluid control device 472, to the patient P or another suitable outflow location. The fluid control device 472 can include a one-way valve, a check valve, or any other suitable fluid control device. A first (e.g., distal) end of the fluid control device 472 is fluidly coupled to the valve 258 of the filtering syringe 230, and a second (e.g., proximal) end of the fluid control device 472 is fluidly coupled to the tubing section 474. In the illustrated embodiment, the fluid control device 472 is configured to allow fluid (e.g., the first material FM, FIG. 3) to flow in the second direction D2 (e.g., through the fluid control device 472 to and/or toward the patient P), and to substantially or fully prevent fluid from flowing in the first direction D1 (e.g., opposite the second direction D2, from the patient P toward the filtering syringe 230).

In operation, the user can couple the filtering syringe 230 to the assembly 10 (e.g., via the connector 128, FIG. 1) and actuate the assembly 10 to aspirate the aspirated material AM from the patient P, as described previously herein. Then, the user can close the fluid control device 126 (FIG. 1) and move the plunger 252 in the first direction D1 to filter the aspirated material AM. Specifically, movement of the plunger 252 in the first direction D1 drives the first material FM in the second direction D2, through the filter assembly 260, the lumen 256, and the valve 258 and into the filtered material subsystem 470. In some embodiments, at least a portion of the first material FM can be returned to/reintroduced into the patient P via the tubing 474.

In some aspects of the present technology, the filtering syringe 230 can remain coupled to the filtered material subsystem 470 for one or more aspiration cycles, e.g., unless or until the filtering syringe 230 is filled with the second material UFM (FIG. 3) and/or the user determines that the filtering syringe 230 should be cleaned/flushed. Accordingly, in at least some aspects of the present technology, the filtering syringe 230 and the filtered material subsystem 470 are expected to form a generally or substantially closed-loop system operable to remove aspirated material from the patient and return at least a portion of the aspirated material (e.g., the first material FM) to the patient P.

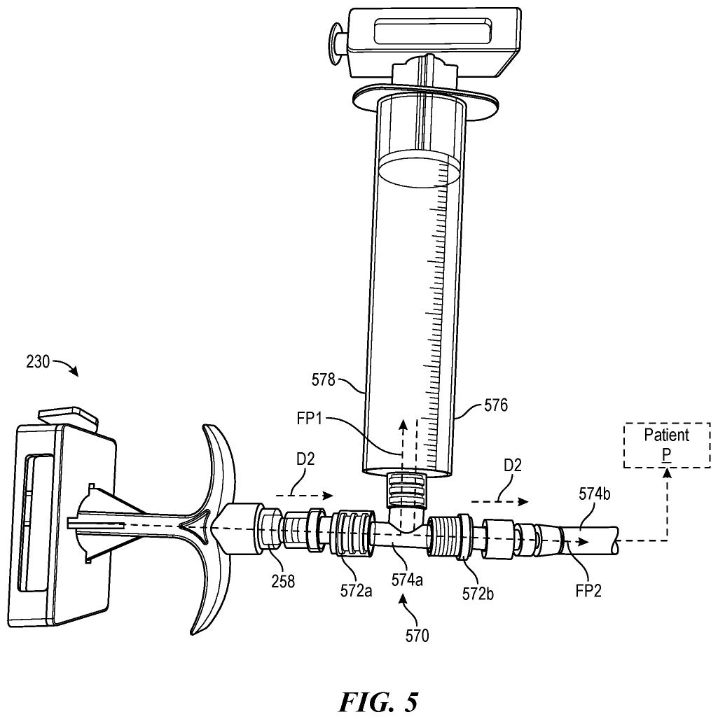

FIG. 5 is a side view of the filtering syringe 230 of FIG. 2A coupled to a filtered material subsystem 570 in accordance with embodiments of the present technology. In the illustrated embodiment, the filtered material subsystem 570 includes two fluid control devices 572a-b (i.e., a first fluid control device 572a and a second fluid control device 572b), two tubing sections 574a-b (i.e., a first tubing section 574a and a second tubing section 574b), and a fluid drive component 576. Each of the fluid control devices 572a-b can be generally similar or identical in structure and/or function to the fluid control device 472 of FIG. 4. The fluid drive component 576 can include a syringe (e.g., “the drive syringe 576”), which can be generally similar or identical in structure and/or function to the syringe 370 of FIG. 3. The first fluid control device 572a can be coupled to the valve 258 of the filtering syringe 230; the first tubing section 574a can be (i) positioned between the first fluid control device 572a and the second fluid control device 572b, and (ii) fluidly coupled to the drive syringe 576; and the second tubing section 574b can be fluidly coupled to the second fluid control device 572b opposite the first tubing section 574a. In the illustrated embodiment, the first tubing section 574a is a “T-shaped” tubing section and the drive syringe 576 has a plunger 578 that can be withdrawn/depressed to drive fluid in/out of the drive syringe 576.

The first and second fluid control devices 572a-b are each one-way valves positioned to allow fluid (e.g., the first material FM, FIG. 3) to flow in the second direction D2 (e.g., from the filtering syringe 230 toward the patient P), and to substantially or fully prevent the fluid from flowing in a direction opposite the second direction D2 (e.g., from the patient P toward the filtering syringe 230). Accordingly, the first and second fluid control devices 572a-b can define one or more flow paths FP (i.e., a first flow path FP1 and a second flow path FP2) through the filtered material subsystem 570. The drive syringe 576 is configured to control the flow of fluid through the filtered material subsystem 570, e.g., through the first and second flow paths FP1, FP2. In the illustrated embodiment, for example, the user can withdraw the plunger 578 to drive fluid along the first flow path FP1 from the filtering syringe 230, through the first fluid control device 572a, and into the drive syringe 576. As the user withdraws the plunger 578, the second fluid control device 572b can substantially or fully prevent the drive syringe 576 from drawing fluid from the patient P, (e.g., because the force/pressure applied to the second fluid control device 572b when the plunger 578 is withdrawn in a direction opposite the second direction D2).

After the user has withdrawn the plunger 578, the user can depress the plunger 578 to drive fluid along the second flow path FP2 from the drive syringe 576, through the second fluid control device 572b, into the second tubing section 574b, and toward the patient P. As the user depresses the plunger 578, the first fluid control device 572a can substantially or fully prevent the drive syringe 576 from driving fluid flow into the filtering syringe 230 (e.g., because the force/pressure applied to the first fluid control device 572a when the user depresses the plunger 578 is in a direction opposite the second direction D2). In some aspects of the present technology, this can inhibit or prevent filtered material (e.g., the first material FM) from being reintroduced into the filtering syringe 230 and/or the aspirated material AM in the filtering syringe 230 from being driven back into the patient P (e.g., through the catheter 102; FIG. 1).

In some aspects of the present technology, the user can operate the drive syringe 576 to de-air the filtered material subsystem 570 and/or the filtering syringe 230. De-airing the filtering syringe 230 can be generally similar to the filtering process described above, but with the drive syringe 576 drawing air and/or other fluid(s) from the filtering syringe 230 instead of the first material FM. In further aspects of the present technology, after the user has withdrawn the plunger 578 but before or instead of depressing the plunger 578, the user can (i) decouple the drive syringe 576 from the first tubing section 574a, (ii) perform one or more tests on the material within the drive syringe 576, (iii) transfer all or part of the material within the drive syringe 576 to another site and/or patient, (iv) take one or more images of the material within the drive syringe 576, and/or flush the filtering syringe 230 and/or the drive syringe 576.

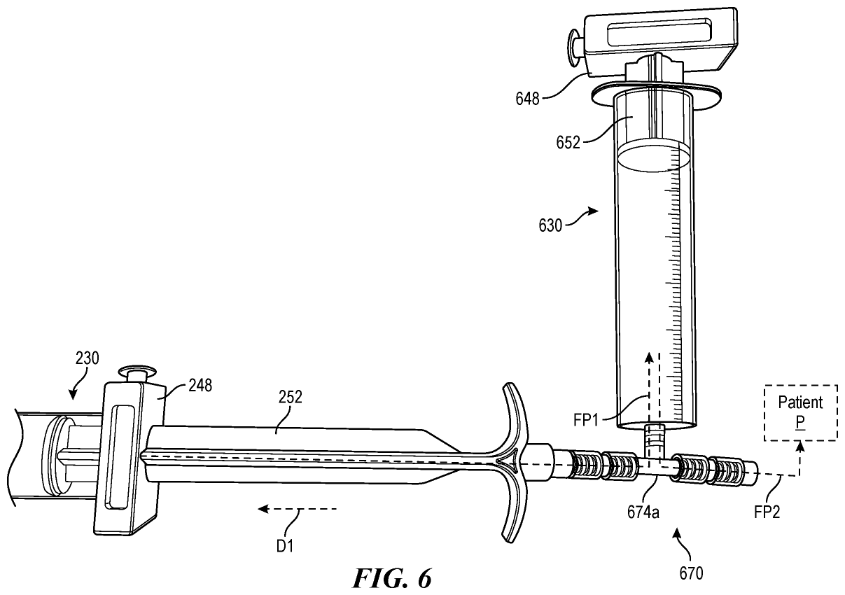

FIG. 6 is a side view of the filtering syringe 230 of FIG. 2A coupled to a filtered material subsystem 670 in accordance with embodiments of the present technology. The filtered material subsystem 670 can include aspects generally similar in structure and/or function to the filtered material subsystem 570 of FIG. 5, with like reference numbers (e.g., first tubing section 674a versus the first tubing section 574a of FIG. 5) indicating like elements. Additionally, the filtered material subsystem 670 includes a second locking syringe 630 (“second syringe 630”) that can be generally similar in structure and/or function to the filtering syringe 230 (“first filtering syringe 230”) of FIG. 2A. In some embodiments, the second syringe 630 may not include a filtering assembly or filters.

In operation, a plunger 652 of the second syringe 630 can be withdrawn and then locked to generate and store vacuum within the second syringe 630. Once material is aspirated into the filtering syringe 230, the vacuum pressure in the second syringe 630 can automatically draw some of the aspirated material toward and through the filter assembly 260 along the first flow path FP1 such that some of the filtered blood can be drawn into the second syringe 630. To fully filter the material from the filtering syringe 230 into the filtered material subsystem 670, the locking component 248 can be unlocked to allow the plunger 252 to move in the first direction D1 to equalize the pressures between the filtering syringe 230 and the second syringe 630. After the filtered material FM is drawn into the second syringe 630, the second plunger 652 can be unlocked (via, e.g., locking component 648) and depressed to drive fluid from the second syringe 630 along the second flow path FP2 from the second syringe 630 to and/or toward the patient P.

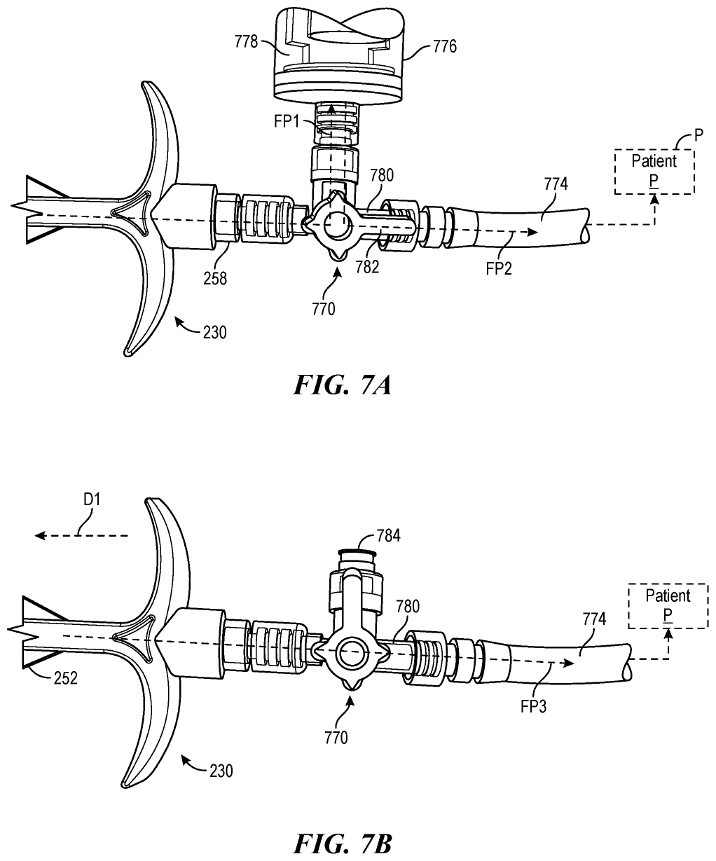

FIG. 7A is a side view of the filtering syringe 230 of FIG. 2A coupled to a filtered material subsystem 770 in accordance with embodiments of the present technology. The filtered material subsystem 770 can include aspects that are generally similar in structure and/or function to the filtered material subsystem 570 of FIG. 5, with like reference numbers (e.g., drive syringe 776 versus the drive syringe 576 of FIG. 5) indicating like elements. Additionally, the filtered material subsystem 770 includes a fluid control device 780. The fluid control device 780 can include a three-way valve, a stopcock, or any other suitable fluid control device. Each of the filtering syringe 230, the drive syringe 776, and tubing section 774 can be fluidly coupled to the fluid control device 780. The fluid control device 780 can be configured to selectively control the flow of fluid through one or more of the flow paths FP1, FP2 within the filtered material subsystem 770. In the illustrated embodiment, for example, the fluid control device 780 includes an actuation element or lever 782 operable to transition the fluid control device 780 between (i) a first configuration (shown in FIG. 7A) in which the first flow path FP1 is open (e.g., the fluid control device 780 allows fluid to flow from the filtering syringe 230 toward and/or to the drive syringe 776) and the second flow path FP2 is closed (e.g., the fluid control device 780 partially or fully prevents fluid from entering the tubing section 774), and (ii) a second configuration (not shown) in which the first flow path FP1 is closed (e.g., the fluid control device 780 partially or fully prevents fluid from entering the filtering syringe 230) and the second flow path FP2 is open (e.g., the fluid control device 780 allows fluid to flow from the drive syringe 776 toward and/or into the tubing section 774).

In operation, the user can transition the fluid control device 780 to the first configuration and withdraw the plunger 778 to drive fluid along the first flow path FP1, e.g., from the filtering syringe 230, through the fluid control device 780, and into the drive syringe 776. As the user withdraws the plunger 778, the fluid control device 780 can substantially or fully prevent the drive syringe 776 from drawing fluid from the patient P when the tubing section 774 is fluidly coupled to the patient P (e.g., because the second flow path FP2 is closed when the fluid control device 780 is in the first configuration). After the user has withdrawn the plunger 778, the user can transition the fluid control device 780 from the first configuration toward and/or to the second configuration and depress the plunger 778 to drive fluid along the second flow path FP2 from the drive syringe 776, through the fluid control device 780, into the tubing section 774, and toward the patient P. As the user depresses the plunger 778, the fluid control device 780 can substantially or fully prevent the drive syringe 776 from driving fluid into the filtering syringe 230 because the first flow path FP1 is closed when the stopcock is in the second configuration. In some embodiments, after depressing the plunger 778, the user can perform one or more additional aspiration cycles with the filtered material subsystem 770 without disconnecting the filtering syringe 230 or the drive syringe 776 from the fluid control device 780.

FIG. 7B illustrates the filtering syringe 230 of FIG. 2A coupled to the filtered material subsystem 770 in accordance with additional embodiments of the present technology. In FIG. 7B, the drive syringe 776 (FIG. 7A) has been disconnected from the fluid control device 780 such that the fluid control device 780 has a free aperture or port 784. Additionally, the fluid control device 780 has been transitioned to a third configuration in which fluid can flow along a third flow path FP3, from the filtering syringe 230, through the fluid control device 780, and toward the patient P. Accordingly, when the fluid control device 780 is in the third configuration, the user can drive fluid through the filtered material subsystem 770 by moving the plunger 252 in the first direction D1, as described in detail above with reference to FIG. 4. In some aspects of the present technology, the user can de-air/flush the filtering syringe 230 and/or the tubing section 774 via the free port 784. For example, the user can transition the fluid control device 780 to the first configuration (FIG. 7A) to de-air/flush the filtering syringe 230 along the first flow path FP1 (FIG. 7A) and/or transition the fluid control device 780 to the second configuration (not shown) to de-air/flush the tubing section 774 along the second flow path FP2 (FIG. 7A).

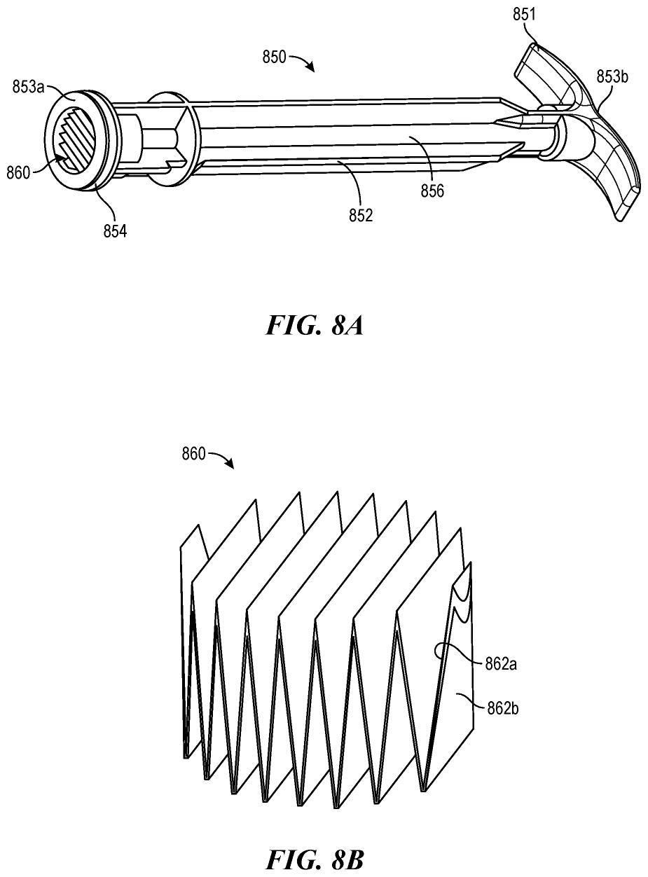

FIG. 8A is a perspective view of another plunger assembly 850 configured in accordance with embodiments of the present technology. The plunger assembly 850 can include at least some aspects that are generally similar or identical in structure and/or function to the plunger assembly 250 of FIGS. 2A-2C, with like reference numbers (e.g., lumen 856 versus the lumen 256 of FIGS. 2A-2C) indicating generally similar or identical aspects. Additionally, in the illustrated embodiment, the plunger assembly 250 further includes a pleated filter assembly 860. Referring to FIG. 8B, in the illustrated embodiment the pleated filter assembly 860 includes a first filter 862a and a second filter 862b. In other embodiments, the pleated filter assembly 860 can include more or fewer filters. In these and other embodiments, the pleated filter assembly 860 can have a greater filtering surface area compared to the generally flat or planar filters/filter assemblies. Accordingly, in some aspects of the present technology, the pleated filter assembly 860 can have an increased filtering surface area compared to the filter assembly 260 of FIGS. 2A-2C while having a generally similar or identical cross-sectional area as the filter assembly 260.

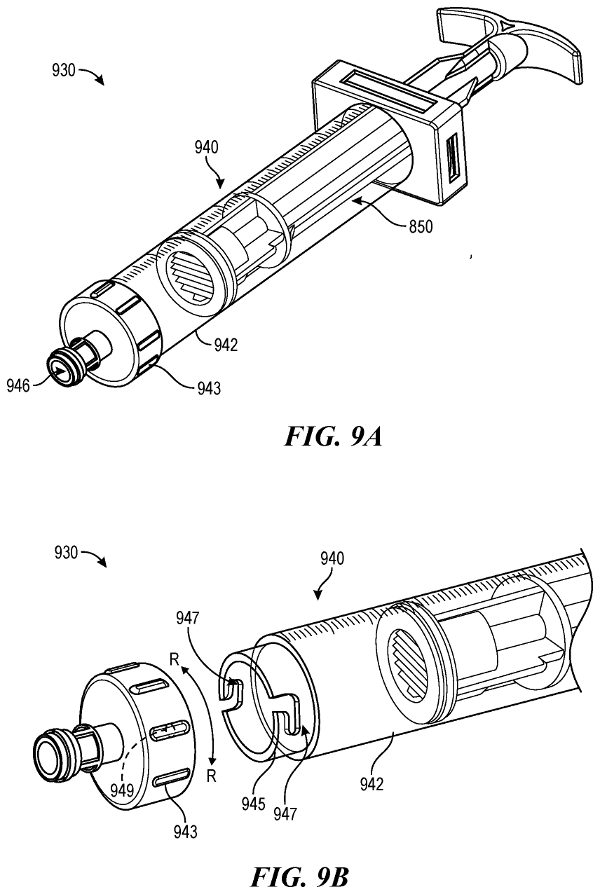

FIGS. 9A and 9B are perspective views of another filtering syringe 930 configured in accordance with embodiments of the present technology. The filtering syringe 930 can include at least some aspects that are generally similar or identical in structure and/or function to the filtering syringe 230 of FIGS. 2A-2C, with like reference numbers (e.g., lumen 946 versus the lumen 246 of FIG. 2A) indicating generally similar or identical aspects. In the illustrated embodiment, the filtering syringe 930 includes the plunger assembly 850 of FIG. 8A. In other embodiments, the filtering syringe 930 can include the plunger assembly 250 of FIGS. 2A-2C, or any other suitable plunger assembly. Additionally, the filtering syringe 930 includes a detachable cap 943 (which can also be referred to as a “locking cap,” a “twist cap,” a “removable cap,” and the like). The detachable cap 943 can be releasably coupled to the barrel 242. Referring to FIG. 9B, the detachable cap 943 can be configured to matingly engage with a coupling portion 945 of the barrel 242. In the illustrated embodiment, the coupling portion 945 includes one or more coupling slots or grooves 947, the detachable cap 943 includes one or more tabs or protrusions 949 (which can extend radially from an inner surface of the cap 943, as shown in FIG. 9B), and each coupling slot 947 is configured to slidably receive a corresponding tab 949 to couple the detachable cap 943 to the barrel 942. In at least some embodiments, the detachable cap 943 can include an O-ring or other sealing element (not visible in FIG. 9B) that forms a substantially fluid-impermeable seal with the barrel 942 when the detachable cap 943 is coupled to the barrel 942, for example, to maintain a vacuum pressure generated/stored within the barrel 942, and/or to inhibit or prevent fluid and/or other aspirated material within the barrel 942 from leaking out the barrel 942.

In operation, a user can rotate the detachable cap 943 relative to the barrel 942 (e.g., in the direction shown by the arrow R) to couple and/or uncouple the detachable cap 943 and the barrel 942. The user can uncouple the detachable cap 943 from the barrel 942 after performing one or more material removal cycles to, for example, flush or remove any fluid, clot material, or other aspirated material collected within the barrel 942. After flushing/removing material collected within the barrel 942, the user can recouple the detachable cap 943 to the barrel 942 and use the filtering syringe 930 to perform one or more additional material removal cycles, alone or in combination with any of the filtered material systems described in detail with reference to FIGS. 3-7B.

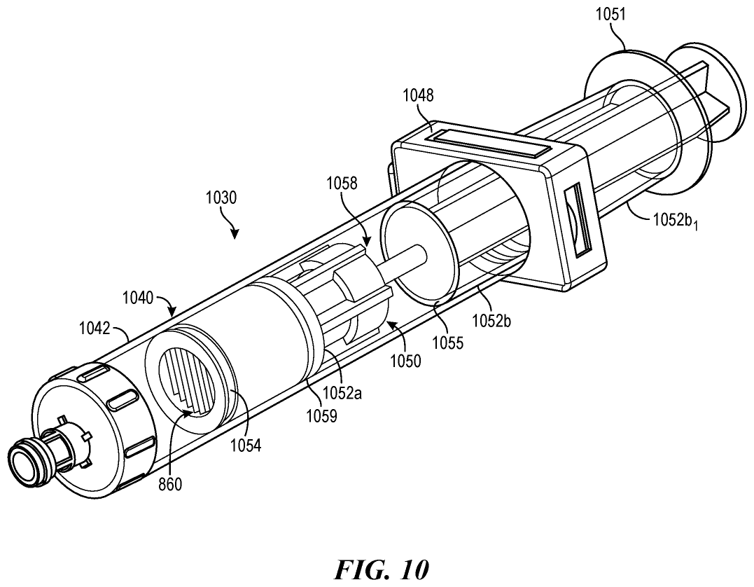

FIG. 10 is a perspective view of another filtering syringe 1030 configured in accordance with embodiments of the present technology. The filtering syringe 1030 can include at least some aspects that are generally similar or identical in structure and/or function to the filtering syringe 230 of FIG. 2A-2C and/or the filtering syringe 930 of FIGS. 9A-9B, with like numbers (e.g., barrel 1042 versus the barrel 242 of FIG. 2A and/or the barrel 942 of FIGS. 9A and 9B) indicating generally similar or identical aspects. The filtering syringe 1030 can include a plunger assembly 1050 having a first (e.g., distal) body portion 1052a and a second (e.g., proximal) body portion 1052b. The first body portion 1052a can include the filter assembly 860 of FIGS. 8A and 8B, a seal or sealing element 1054, a valve 1058 (e.g., a luer-activated valve, a one-way valve, or the like), and/or a locking feature 1059 configured to engage a locking component 1048 of the filtering syringe 1030. In other embodiments, the first body portion 1052a can include the filter assembly 260 of FIGS. 2A-2C, or any other suitable filter and/or filter assembly. The second body portion 1052b can include a handle 1051, a plunger 1052b1, and/or a tip 1055. The tip 1055 can be configured to couple (e.g., fluidly coupled) to the valve 1058, such that the second body portion 1052b can be coupled/connected to the first body portion 1052a.

In the illustrated embodiment, the second body portion 1052b includes a syringe (“syringe 1052b”), such as a 30 cc luer-lock syringe, or any other suitable syringe, including any of the filtering syringes described herein. In these and other embodiments, the syringe 1052b can have an internal volume less than, equal to, or greater than an internal volume of the barrel 1042. The tip 1055 of the syringe 1052b can be coupled to the first body portion 1052a, such that movement of the syringe 1052b can cause corresponding movement of the first body portion 1052a. In operation, for example, the plunger assembly 1050 (e.g., the syringe 1052b) can be drawn (e.g., proximally, by a user) relative to the barrel 1042 to generate and/or store a vacuum within the filtering syringe 1030, as described previously with reference to FIGS. 1-2C. Continuing to move the plunger assembly 1050 can cause the locking component 1048 to engage the locking feature 1059 of the first body portion 1052a, for example, to lock the plunger assembly 1050 relative to the barrel assembly 1040 and store a vacuum within the barrel 1042, as described previously regarding FIGS. 2A-2C. Once a vacuum has been generated and/or stored within the filtering syringe 1030, the filtering syringe 1030 can be used to perform one or more material removal cycles, for example, as described previously with reference to FIGS. 1-2C, and/or alone or in combination with any of the filtered material systems described in detail with reference to FIGS. 3-7B. After the filtering syringe 1030 has been used to perform a material removal cycle, at least a portion of the aspirated material within the barrel 1042 can be filtered by actuating the plunger 1052b1 of the syringe 1052b to draw material (e.g., blood) through the filter assembly 860 and into the syringe 1052b via the valve 1058. In at least some embodiments, the syringe 1052b can be configured such that all or substantially all of the aspirated blood within the barrel 1042 can be filtered and drawn into the syringe 1052b. Additionally, or alternatively, the syringe 1052b can be advanced toward the aspirated material within the barrel 1042 to filter material within the barrel 1042. In these and other embodiments, because the first body portion 1052a sealingly engages the barrel 1042, withdrawing the plunger 1052b1 proximally can cause a corresponding distal movement of the syringe 1052b and the first body portion 1052a within the barrel 1042 as blood is filtered and drawn into the syringe 1052b. Similarly, moving the syringe 1052b distally can cause a corresponding proximal movement of the plunger 1052b1. Once at least a portion of the filtered material is drawn into the syringe 1052b, the syringe 1052b can then be disconnected from the first body portion 1052a and the filtered material (e.g., blood) within the syringe 1052b can be, for example, returned to the patient.

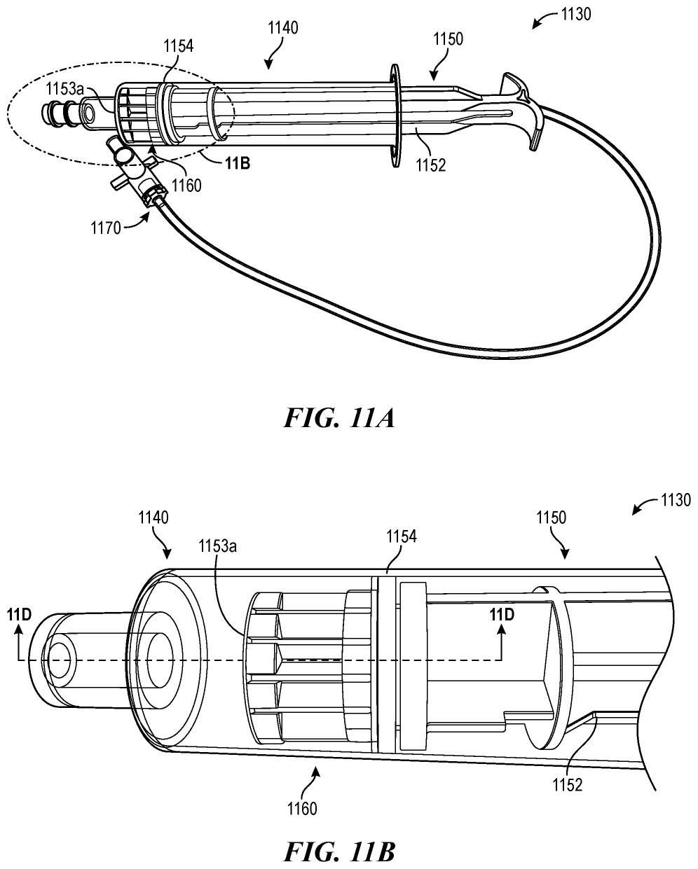

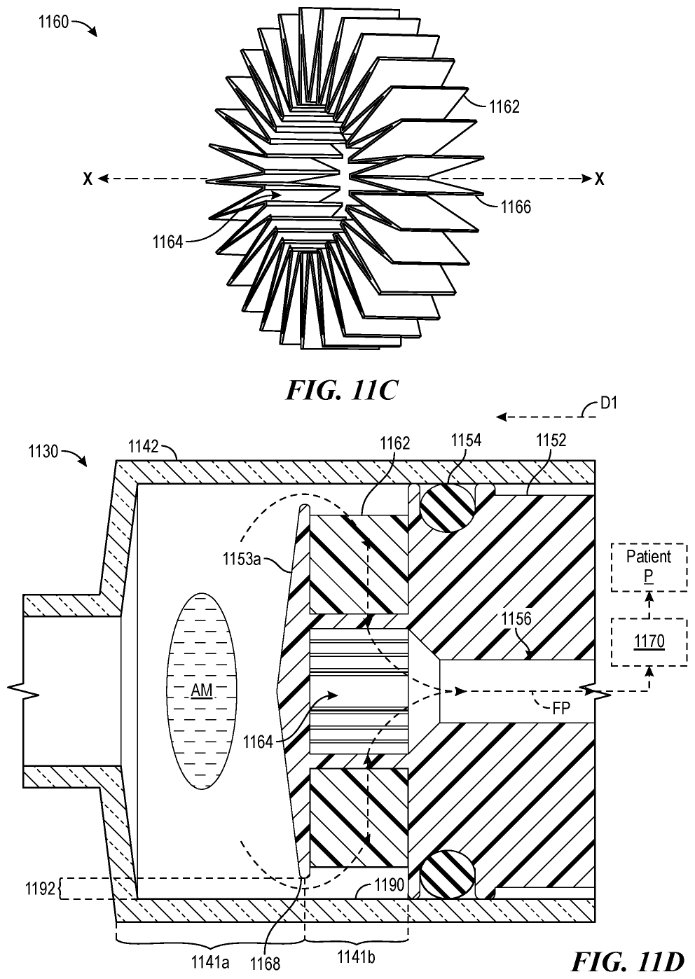

FIG. 11A is a perspective view of another filtering syringe 1130 configured in accordance with embodiments of the present technology. FIG. 11B is an enlarged perspective view of a distal portion 11B of the filtering syringe 1130 in accordance with embodiments of the present technology. Referring to FIGS. 11A and 11B together, the filtering syringe 1130 can include at least some aspects that are generally similar or identical in structure and/or function to the filtering syringe 230 of FIG. 2A-2C, the filtering syringe 930 of FIGS. 9A-9B, and/or the filtering syringe 1030 of FIG. 10, with like numbers (e.g., barrel assembly 1140 versus the barrel assembly 240 of FIG. 2A, the barrel assembly 940 of FIGS. 9A and 9B, and/or the barrel assembly 1040 of FIG. 10) indicating generally similar or identical aspects. The filtering syringe 1130 can include the barrel assembly 1140, a plunger assembly 1150, and a filtered material subsystem 1170. The filtered material subsystem 1170 can be fluidly coupled to the plunger assembly 1150. The plunger assembly 1150 can include a plunger 1152 that can be slidably received within the barrel assembly 1140. The plunger 1152 can include a filter assembly 1160 and a seal 1154. In the illustrated embodiment, the filter assembly 1160 is seated at least partially within the plunger 1152 proximal to a first or distal end portion 1153a of the plunger 1152 and distal from the seal 1154. In other embodiments, the filter assembly 1160 can have another suitable position.

FIG. 11C is a perspective view of the filter assembly 1160 in accordance with embodiments of the present technology. The filter assembly 1160 can include an annular filter 1162 defining a central space 1164. The annular filter 1162 can be configured to allow flow of select fluids (e.g., blood) across the annular filter 1162 toward and/or away from the central space 1164, such as radially outwardly or inwardly relative to a longitudinal axis X of the annular filter 1162. In the illustrated embodiment, the annular filter 1162 includes one or more folds or pleats 1166, each of which can increase a surface area of the annular filter 1162 while maintaining a generally compact formfactor. In some aspects of the present technology, the pleats 1166 and/or the increased surface area of the annular filter 1162 associated therewith are expected to reduce or prevent blockages, clogs, and/or other reductions of fluid flow through the annular filter 1162, which can increase the number of filtrations performed before replacement of the annular filter 1162.

FIG. 11D is an enlarged side cross-sectional view of the filtering syringe 1130 taken along section line 11D-11D in FIG. 11B in accordance with embodiments of the present technology. The first end portion 1153a of the plunger 1152 can at least partially define a first or distal chamber portion 1141a on a first (e.g., distal) side of the first end portion 1153a and a second or proximal portion chamber portion 1141b on a second (e.g., proximal) side of the first end portion 1153a opposite the first side. A lateral-most edge 1168 of the first end portion 1153a can have a dimension (e.g., a diameter) less than a corresponding dimension (e.g., a dimeter) of an inner surface 1190 of the barrel 1142, such that the lateral-most edge 1168 can be spaced apart from an inner surface 1190 of the barrel 1142 to define a gap or fluid passage 1192 that fluidly couples the first chamber portion 1141a and the second chamber portion 1141b. The annular filter 1162 can be carried by the plunger 1152 and can be positioned at least partially within the second chamber portion 1141b, such as with the central space 1164 of the annular filter 1162 at least partially aligned with a lumen 1156 of the plunger 1152, and/or such that all or substantially all fluid within the barrel 1142 that enters the lumen 1156 must pass through the annular filter 1162. In the illustrated embodiment, the annular filter 1162 is radially inward from the lateral-most edge 1168, such that the lateral-most edge 1168 overhangs or otherwise extends radially outward relative to the annular filter 1162.

During a procedure, aspirated material AM including blood and/or clot material can be drawn into the first chamber portion 1141a of the barrel 1142. The plunger 1152 can be advanced in the first direction D1 and/or a vacuum can be applied to the lumen 1156 to cause at least some material (e.g., blood) within the first chamber portion 1141a to flow through the gap 1192, into the second chamber portion 1141b, at least partially through the annular filter 1162, into the central space 1164 of the annular filter 1162, and/or into the lumen 1156, such as shown by flow path arrow FP in FIG. 11D. The material (e.g., blood) within the lumen 1156 can flow into the filtered material subsystem 1170 and/or be returned to the patient P, including via any of the techniques described previously herein. The first end portion 1153a and/or the gap 1192 between the first chamber portion 1141a and the second chamber portion 1141b are expected to prevent at least a portion of the aspirated material AM (e.g., at least some aspirated clot material) from entering the second chamber portion 1141b and/or otherwise reduce or prevent contact between the aspirated material AM and the annular filter 1162, without or substantially without inhibiting the flow of other material (e.g., blood) between the first chamber portion 1141a and the second chamber portion 1141b. This, in turn, is expected to reduce or prevent clogs or other obstructions to fluid flow through the annular filter 1162. Accordingly, the first end portion 1153a and/or the gap 1192 can be configured to provide a first filtering stage that inhibits or prevents relatively large material from entering the second chamber portion 1141b while the annular filter 1162 can provide a second filtering stage that inhibits or prevents relatively smaller material from entering the lumen 1156.

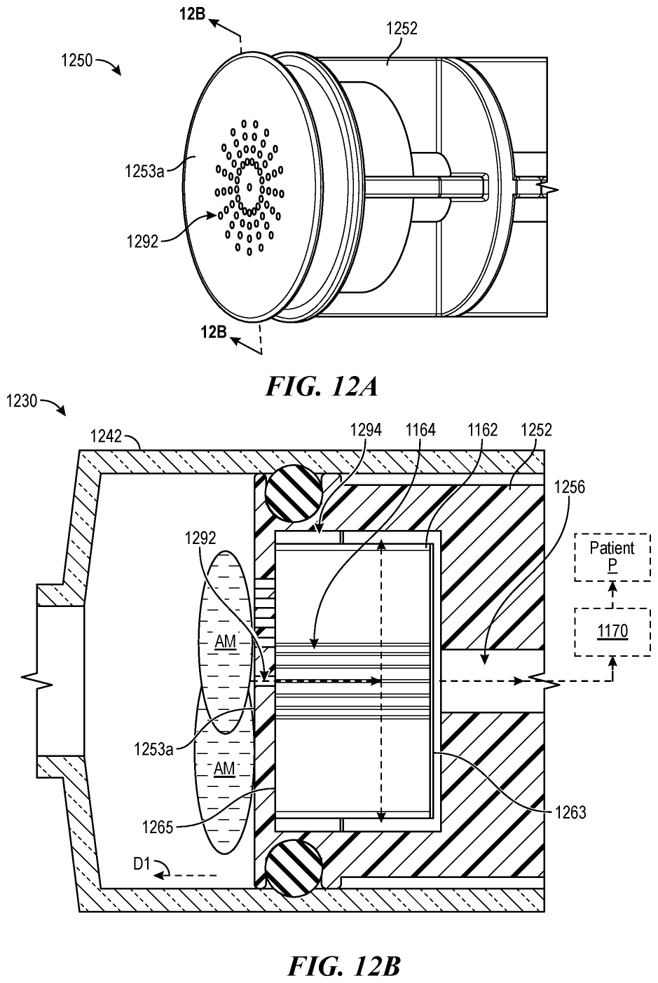

FIG. 12A is a perspective view of a plunger assembly 1250 of another filtering syringe configured in accordance with embodiments of the present technology. At least some aspects of the plunger assembly 1250 can be at least generally similar or identical in structure and/or function to one or more of the plunger assemblies 250, 850, 1050, 1150 described previously herein. For example, the plunger assembly 1250 includes a plunger 1252 configured to carry one or more filters 1162 (FIG. 12B). However, a first or distal end portion 1253a of the plunger 1252 includes one or more holes or apertures 1292.

FIG. 12B is a cross-sectional view of the plunger assembly 1250 of FIG. 12A taken along section line 12B-12B in FIG. 11B. In FIG. 12B, the plunger assembly 1250 is shown positioned within a barrel 1242 of a filtering syringe 1230, in accordance with embodiments of the present technology. As best seen in FIG. 12B, the plunger 1252 defines a filter chamber 1294 configured to receive the annular filter 1162 and a lumen 1256 that can be fluidly coupled to the filtered material subsystem 1170 and/or the patient. The holes 1292 formed in the first end portion 1253a of the plunger 1252 can be configured to allow fluid communication between the filter chamber 1294 (e.g., the central space 1164 of the annular filter 1162) and the exterior of the plunger 1252 (e.g., the interior of the barrel 1242). More specifically, when the annular filter 1162 is positioned within the filter chamber 1294, individual ones of the holes 1292 can allow at least some of the aspirated material AM within the barrel 1242 (including, e.g., blood and/or other fluids) to flow into the central space 1164 of the annular filter 1162. A proximal end portion 1263 of the filter 1162 can be sealed so that at least some of the aspirated material AM that passes through the holes 1292 is caught within the central space 1164, e.g., and does not pass through the filter 1162 into the filter chamber 1294 and/or the lumen 1256. A distal end portion 1265 of the filter 1162 can be coupled to at least part of the first end portion 1253a, e.g., to prevent, or at least partially prevent, the aspirated material AM from entering the filter chamber 1294 without passing through the central space 1164 of the annular filter 1162.

During a procedure, the plunger 1252 can be moved the first direction D1 relative to the barrel 1242 to force at least some of the aspirated material AM through individual ones of the holes 1292 and/or into the central space 1164. As the aspirated material AM collects within the central space 1164, the filter 1162 can separate blood and/or other fluids contained within the aspirated material AM from the aspirated material AM. Because the proximal and distal end portions 1263, 1265 of the annular filter 1162 are sealed, the blood and/or other fluids can be separated by causing the blood and/or other fluids to flow outwardly from the central space 1164 across the filter 1162. For example, moving the plunger 1252 in the first direction D1 relative to the barrel 1242 can force the blood and/or other fluids across the filter 1162, into a space between the filter 1162 and the filter chamber 1294, and into and/or through at least a portion of the lumen 1256. The blood and/or other fluids within the lumen 1256 can flow to the filtered material subsystem 1170 and/or be returned to the patient P.

Accordingly, in some aspects of the present technology, a filtering syringe can be coupled to a catheter subsystem insertable within a patient. The filtering syringe can aspirate material (e.g., clot material, blood, etc.) from a patient via the catheter subsystem. The filtering syringe can include a plunger assembly, and the plunger assembly can include one or more filters. Individual ones of the filters can be configured to (i) allow at least a first portion of aspirated material to pass through at least one of the filters, and (ii) prevent at least a second portion of the aspirated material from passing through at least one of the filters. In some embodiments, for example, the filters are configured to (i) allow blood to pass through the filters, and (ii) prevent clot material from passing through the filters, such that the filters can “filter” blood from the other material aspirated from the patient. In some embodiments, the filtering syringe is fluidly coupled to a filtered material subsystem. The filtered material subsystem can include one or more valves and/or tubing sections that can fluidly couple the filtering syringe to the patient or another suitable fluid (e.g., blood) outflow location. The filtered material subsystem can (i) capture/receive all or part of the first portion of the aspirated material filtered by the filtering syringe, and/or (ii) return/reintroduce all or part of the first portion of the aspirated material into the patient.

Several aspects of the present technology are set forth in the following examples:

1. A filtering syringe for treatment of clot material from within a blood vessel of a patient, the filtering syringe comprising:
a barrel defining a chamber configured to receive at least a portion of the clot material and blood from the human patient;
a plunger assembly having a first end portion and a second end portion opposite the first end portion, wherein the plunger assembly includes a filter proximate to the first end portion, a port proximate to the second end portion, and a lumen therebetween and fluidly coupling the filter and the port;
wherein at least the first end portion of the plunger assembly is insertable within the chamber and slidable relative to the barrel; and
wherein the filter is configured to allow at least part of the blood received within the chamber to separate from the portion of the clot material and enter the lumen of the plunger assembly.

2. The filtering syringe of example 1, further comprising a tip configured to fluidly couple the filtering syringe to a clot treatment system.

3. The filtering syringe of example 1 or example 2 wherein the filter has a circular, annular, cylindrical, and/or pleated shape.

4. The filtering syringe of any of examples 1-3 wherein the filter has a porosity between about 40 microns and about 200 microns.

5. The filtering syringe of any of examples 1-4 wherein the filter includes at least one of a polyester, a polyamide, and/or a hemocompatible material.

6. The filtering syringe of any of examples 1-5 wherein the filter is a first filter having a first porosity, the filtering syringe further comprising a second filter having a second porosity.

7. The filtering syringe of example 6 wherein the first porosity is different than the first porosity.

8. The filtering syringe of example 6 wherein the first porosity is the same as the second porosity.

9. The filtering syringe of any of examples 6-8 wherein the second filter is positioned proximate the first end portion of the plunger assembly.

10. The filtering syringe of any of examples 6-9 wherein the second filter is spaced apart from the first filter.

11. The filtering syringe of any of examples 1-10 wherein the port includes a luer-activated valve.

12. The filtering syringe of any of examples 1-11 wherein the first end portion of the plunger assembly is a distal end portion of the plunger assembly and the second end portion is a proximal end portion of the plunger assembly, wherein the filter is positioned proximally from the distal end portion.

13. The filtering syringe of any of examples 1-12 wherein the chamber includes an inner surface, and wherein first end portion of the plunger assembly is radially spaced apart from chamber such that the barrel assembly and the plunger assembly at least partially define (i) a first chamber portion positioned on a first side of the first end portion of the plunger assembly, (ii) a second chamber portion positioned on a second side of the first end portion of the plunger assembly, the second side opposite the first side, and (iii) a gap fluidly coupling the first chamber portion and the second chamber portion.

14. The filtering syringe of example 13 wherein the filter is positioned in the second chamber portion, wherein the first chamber portion is configured to receive at least the portion of the clot material from the patient, wherein the gap is configured to at least partially prevent the portion of the clot material from entering the second chamber portion, and wherein the filter is configured to allow blood within the second chamber portion to enter the lumen.

15. The filtering syringe of any of examples 1-14 wherein the first end portion of the plunger assembly is configured to at least partially prevent contact between the filter and the portion of the clot material.

16. The filtering syringe of any of examples 1-15 wherein the plunger assembly defines a filter chamber configured to receive the filter, and wherein the first end portion of the plunger assembly includes one or more holes in fluid communication with the filter chamber.

17. The filtering syringe of example 16 wherein the filter is an annular filter defining a central space, and wherein individual ones of the one or more holes are positioned to direct at least a portion of the clot material and/or the blood received from the patient into the central space.

18. A system for intravascular treatment of clot material from within a blood vessel of a human patient, the system comprising:
a catheter configured to be intravascularly positioned at a treatment site proximate to the clot material within the blood vessel;
a filtering syringe configured to generate negative pressure, wherein the filtering syringe includes—
   a tip configured to be fluidly coupled to the catheter to receive at least a portion of the clot material and blood from within the blood vessel via the catheter, and
   a filter assembly including one or more filters configured to filter the blood from the portion of the clot material.

19. The system of example 18, further comprising a filtered material system fluidly coupled to the filtering syringe and configured to receive at least a portion of the filtered blood.

20. The system of example 19, wherein the filtered material system includes a fluid drive component operable to drive flow of the filtered blood through at least part of the filtered material system.

21. The system of example 20 wherein the filtered material system includes at least one flow path, and wherein fluid drive component is operable to drive the flow of the filtered blood through the at least one flow path.

22. The system of example 21 wherein the at least one flow path is configured to direct fluid flow away from the filtering syringe.

23. The system of any of examples 19-22 wherein the filtering syringe further includes a valve opposite the tip, wherein the filtered material system includes (i) a fluid control device fluidly coupled to the valve and (ii) tubing fluidly coupled to the fluid control device, and wherein the fluid control device is configured to at least partially prevent filtered blood within the tubing from entering the filtering syringe.

24. A method for removing clot material from within a blood vessel of a human patient, the method comprising:
positioning a distal portion of a catheter proximate to the clot material within the blood vessel;
coupling a filtering and pressure-generating syringe to the catheter via a fluid control device, wherein (i) opening of the fluid control device fluidly connects the filtering and pressure-generating syringe to the catheter, and (ii) closing the fluid control device fluidly disconnects the filtering and pressure-generating syringe from the catheter;
   activating the syringe to generate a vacuum while the fluid control device is closed;
   opening the fluid control device to apply the vacuum to the catheter to thereby aspirate at least a portion of the clot material into the catheter; and
   filtering blood from the portion of the clot material via a filter of the syringe.

25. The method of example 24 wherein filtering the blood includes moving a plunger of the syringe to cause the filter to move toward the portion of the clot material.

26. The method of example 24 or example 25 wherein filtering the blood includes actuating a fluid drive component of a filtered material system to cause the blood to flow through the filter proximally toward the fluid drive component.

27. A filtering syringe for removing target material from within a human patient, the filtering syringe comprising:
a barrel defining a chamber configured to receive at least a portion of the target material and fluid from the human patient;
a plunger assembly having a first end portion and a second end portion opposite the first end portion, wherein the plunger assembly includes a filter proximate to the first end portion, a valve proximate to the second end portion, and a lumen therebetween and fluidly coupling the filter and the valve;
wherein at least the first end portion of the plunger assembly is insertable within the chamber and slidable relative to the barrel; and
wherein the filter is configured to allow at least part of the fluid received within the chamber to pass through the filter and enter the lumen of the plunger assembly.

28. The filtering syringe of example 27 wherein the fluid includes blood.

29. The filtering syringe of example 27 or example 28 wherein the target material includes clot material.

30. A filtering syringe for treating a human patient, comprising:
a barrel defining a chamber configured to receive first material and second material from within the human patient;
a plunger assembly having a first end portion and a second end portion opposite the first end portion, wherein the plunger assembly includes a filter proximate to the first end portion, a valve proximate to the second end portion, and a lumen therebetween and fluidly coupling the filter and the valve;
wherein at least the first end portion of the plunger assembly is insertable within the chamber and slidable relative to the barrel; and
wherein the filter is configured to (i) allow at least part of the first material received within the chamber to pass through the filter and enter the lumen of the plunger assembly, and (ii) prevent at least part of the second material received within the chamber from passing through the filter.

31. The filtering syringe of example 30 wherein the first material includes blood.

32. The filtering syringe of example 30 or example 31 wherein the second material includes clot material.

33. A filtering syringe for treating a human patient, comprising:
a barrel defining a chamber configured to receive first material and second material from within the human patient; and
a plunger assembly including a first body portion and a second body portion, wherein the first body portion includes a filter and a valve, and wherein the second body portion defines an interior chamber and is detachably couplable to the first body portion via the valve;
wherein at least part of the first body portion and the second body portion are insertable within the chamber and slidable relative to the barrel; and
wherein the filter is configured to allow at least part of the first material received within the chamber to pass through the filter and into the interior chamber of the second body portion.

34. The filtering syringe of example 33 wherein the second body portion comprises a syringe including a plunger operable to draw at least part of the first material within the chamber through the filter and into the interior chamber of the syringe.

35. The filtering syringe of example 34 wherein the syringe includes a tip, and wherein the valve of the first body portion is configured to matingly engage the tip of the syringe to fluidly couple the chamber of the barrel to the interior chamber of the syringe.

36. The filtering syringe of any of examples 33-35 wherein the first material includes blood.

37. The filtering syringe of any of examples 33-36 wherein the second material includes clot material.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A filtering syringe for treatment of clot material from within a blood vessel of a patient, the filtering syringe comprising:
a barrel defining a chamber configured to receive at least a portion of the clot material and blood from the patient; and
a plunger assembly having a first end portion and a second end portion opposite the first end portion, wherein the plunger assembly includes a filter proximate to the first end portion, a port proximate to the second end portion, and a lumen therebetween and fluidly coupling the filter and the port;
wherein at least the first end portion of the plunger assembly is insertable within the chamber and slidable relative to the barrel; and
wherein the filter is configured to allow at least part of the blood received within the chamber to separate from the portion of the clot material and enter the lumen of the plunger assembly.

2. The filtering syringe of claim 1, further comprising a tip configured to fluidly couple the filtering syringe to a clot treatment system.

3. The filtering syringe of claim 1 wherein the filter has a circular, annular, cylindrical, and/or pleated shape.

4. The filtering syringe of claim 1 wherein the filter has a porosity between about 40 microns and about 200 microns.

5. The filtering syringe of claim 1 wherein the filter includes at least one of a polyester, a polyamide, and/or a hemocompatible material.

6. The filtering syringe of claim 1 wherein the filter is a first filter having a first porosity, the filtering syringe further comprising a second filter having a second porosity.

7. The filtering syringe of claim 6 wherein the first porosity is different than the first porosity.

8. The filtering syringe of claim 6 wherein the first porosity is the same as the second porosity.

9. The filtering syringe of claim 6 wherein the second filter is positioned proximate the first end portion of the plunger assembly.

10. The filtering syringe of claim 6 wherein the second filter is spaced apart from the first filter.

11. The filtering syringe of claim 1 wherein the port includes a luer-activated valve.

12. The filtering syringe of claim 1 wherein the first end portion of the plunger assembly is a distal end portion of the plunger assembly and the second end portion is a proximal end portion of the plunger assembly, wherein the filter is positioned proximally from the distal end portion.

13. The filtering syringe of claim 1 wherein the chamber includes an inner surface, and wherein first end portion of the plunger assembly is radially spaced apart from chamber such that the barrel assembly and the plunger assembly at least partially define (i) a first chamber portion positioned on a first side of the first end portion of the plunger assembly, (ii) a second chamber portion positioned on a second side of the first end portion of the plunger assembly, the second side opposite the first side, and (iii) a gap fluidly coupling the first chamber portion and the second chamber portion.

14. The filtering syringe of claim 13 wherein the filter is positioned in the second chamber portion, wherein the first chamber portion is configured to receive at least the portion of the clot material from the patient, wherein the gap is configured to at least partially prevent the portion of the clot material from entering the second chamber portion, and wherein the filter is configured to allow blood within the second chamber portion to enter the lumen.

15. The filtering syringe of claim 1 wherein the first end portion of the plunger assembly is configured to at least partially prevent contact between the filter and the portion of the clot material.

16. The filtering syringe of claim 1 wherein the plunger assembly defines a filter chamber configured to receive the filter, and wherein the first end portion of the plunger assembly includes one or more holes in fluid communication with the filter chamber.

17. The filtering syringe of claim 16 wherein the filter is an annular filter defining a central space, and wherein individual ones of the one or more holes are positioned to direct at least a portion of the clot material and/or the blood received from the patient into the central space.

18. A system for intravascular treatment of clot material from within a blood vessel of a human patient, the system comprising:
a catheter configured to be intravascularly positioned at a treatment site proximate to the clot material within the blood vessel; and
a filtering syringe configured to generate negative pressure, wherein the filtering syringe includes—
a tip configured to be fluidly coupled to the catheter to receive at least a portion of the clot material and blood from within the blood vessel via the catheter, and
a filter assembly including one or more filters configured to filter the blood from the portion of the clot material.

19. The system of claim 18, further comprising a filtered material system fluidly coupled to the filtering syringe and configured to receive at least a portion of the filtered blood.

20. The system of claim 19, wherein the filtered material system includes a fluid drive component operable to drive flow of the filtered blood through at least part of the filtered material system.

21. The system of claim 20 wherein the filtered material system includes at least one flow path, and wherein fluid drive component is operable to drive the flow of the filtered blood through the at least one flow path.

22. The system of claim 21 wherein the at least one flow path is configured to direct fluid flow away from the filtering syringe.

23. The system of claim 19 wherein the filtering syringe further includes a valve opposite the tip, wherein the filtered material system includes (i) a fluid control device fluidly coupled to the valve and (ii) tubing fluidly coupled to the fluid control device, and wherein the fluid control device is configured to at least partially prevent filtered blood within the tubing from entering the filtering syringe.

24. A method for removing clot material from within a blood vessel of a human patient, the method comprising:
positioning a distal portion of a catheter proximate to the clot material within the blood vessel;
coupling a filtering and pressure-generating syringe to the catheter via a fluid control device, wherein (i) opening of the fluid control device fluidly connects the filtering and pressure-generating syringe to the catheter, and (ii) closing the fluid control device fluidly disconnects the filtering and pressure-generating syringe from the catheter;
activating the syringe to generate a vacuum while the fluid control device is closed;
opening the fluid control device to apply the vacuum to the catheter to thereby aspirate at least a portion of the clot material into the catheter; and
filtering blood from the portion of the clot material via a filter of the syringe.

25. The method of claim 24 wherein filtering the blood includes moving a plunger of the syringe to cause the filter to move toward the portion of the clot material.

26. The method of claim 24 wherein filtering the blood includes actuating a fluid drive component of a filtered material system to cause the blood to flow through the filter proximally toward the fluid drive component.

27. A filtering syringe for removing target material from within a human patient, the filtering syringe comprising:
a barrel defining a chamber configured to receive at least a portion of the target material and fluid from the human patient; and
a plunger assembly having a first end portion and a second end portion opposite the first end portion, wherein the plunger assembly includes a filter proximate to the first end portion, a valve proximate to the second end portion, and a lumen therebetween and fluidly coupling the filter and the valve;
wherein at least the first end portion of the plunger assembly is insertable within the chamber and slidable relative to the barrel; and
wherein the filter is configured to allow at least part of the fluid received within the chamber to pass through the filter and enter the lumen of the plunger assembly.

28. The filtering syringe of claim 27 wherein the fluid includes blood.

29. The filtering syringe of claim 27 wherein the target material includes clot material.

30. A filtering syringe for treating a human patient, comprising:
a barrel defining a chamber configured to receive first material and second material from within the human patient; and
a plunger assembly having a first end portion and a second end portion opposite the first end portion, wherein the plunger assembly includes a filter proximate to the first end portion, a valve proximate to the second end portion, and a lumen therebetween and fluidly coupling the filter and the valve;
wherein at least the first end portion of the plunger assembly is insertable within the chamber and slidable relative to the barrel; and
wherein the filter is configured to (i) allow at least part of the first material received within the chamber to pass through the filter and enter the lumen of the plunger assembly, and (ii) prevent at least part of the second material received within the chamber from passing through the filter.

31. The filtering syringe of claim 30 wherein the first material includes blood.

32. The filtering syringe of claim 30 wherein the second material includes clot material.

33. A filtering syringe for treating a human patient, comprising:
a barrel defining a chamber configured to receive first material and second material from within the human patient; and
a plunger assembly including a first body portion and a second body portion, wherein the first body portion includes a filter and a valve, and wherein the second body portion defines an interior chamber and is detachably couplable to the first body portion via the valve;
wherein at least part of the first body portion and the second body portion are insertable within the chamber and slidable relative to the barrel; and
wherein the filter is configured to allow at least part of the first material received within the chamber to pass through the filter and into the interior chamber of the second body portion.

34. The filtering syringe of claim 33 wherein the second body portion comprises a syringe including a plunger operable to draw at least part of the first material within the chamber through the filter and into the interior chamber of the syringe.

35. The filtering syringe of claim 34 wherein the syringe includes a tip, and wherein the valve of the first body portion is configured to matingly engage the tip of the syringe to fluidly couple the chamber of the barrel to the interior chamber of the syringe.

36. The filtering syringe of claim 33 wherein the first material includes blood.

37. The filtering syringe of claim 33 wherein the second material includes clot.
